(19) United States
(12) Patent Application Publication
(10) Pub. No.: US 2005/0089442 A1
(43) Pub. Date: Apr. 28, 2005

(54) Method for sterilization using ethylene oxide

(76) Inventors: William Travis Young, Palatine, IL (US); Zhangwu Zhu, Woodridge, IL (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(21) Appl. No.: 10/694,180

(22) Filed: Oct. 28, 2003

(51) Int. Cl. A61L 9/00; A61L 2/08; B01J 19/00; G01D 11/26

(52) U.S. Cl. 422/34; 422/1; 422/4; 422/27; 422/28; 422/32; 422/33; 422/40; 422/105; 422/119

(57) ABSTRACT

A method for sterilizing industrial products using a ethylene oxide in conjunction with one or more of the following techniques is disclosed: steam pulses; steam conditioning; deep vacuum pulses with nitrogen; and, positive pressure pulses of inert gases. The entire sterilization process, which consists of conditioning, sterilizing, and degassing the product, is performed in a singled chamber. The product is releasable from an ethylene oxide residual standpoint at the completion of the process. The entire process takes 10 to 20 hours.

### TECHNICAL FIELD

The technical field includes sterilization, and more specifically industrial sterilization using ethylene oxide.

### BACKGROUND

Gas sterilization is an important process for the manufacture of many industrial products. This is especially true for medical products to be used in a sterile environment.

Gas sterilization is a process for sterilizing items by exposing them to sterilizing gases, e.g. ethylene oxide (EtO or EO), for a period of time. The gas is toxic to biological organisms. To be useful, the process usually involves ensuring that no sterilizing gas residue is left on the article. Conventional gas sterilization is often performed in multiple chambers and can take many days to complete. In such a process, the product is conditioned in one chamber, sterilized (exposed to a sterilent gas) in a different chamber, and finally degassed (removal of the sterilent gas) in yet another chamber. Current single chamber sterilization processes are prolonged and sometimes not as effective as required.

It would be advantageous to simplify the gas sterilization process by reducing the time required for performing a single chamber sterilization while maintaining and enhancing the effectiveness of the process.

### SUMMARY

A method for sterilizing industrial products with gas is disclosed. The method includes the step of conditioning an industrial product to be sterilized by placing the article or product to be sterilized in a chamber, evacuating the chamber, pulsing steam and/or heated inert gas into the chamber, and re-evacuating the chamber. The preferred inert gas is Nitrogen (N₂) heated to a temperature of about 130 to about 170° F. The method further includes the step of injecting a sterilent gas into the chamber. The preferred sterilent gas is ethylene oxide. Next, overpressure of inert gas is introduced into the chamber and maintained while the sterilization reaction occurs, preferably at an incremental pressure of about 5 to about 15 inches of mercury. This period of holding is sometimes referred to as the dwell time. Finally, the product is degassed.

The step of degassing the product may be accomplished by evacuating the chamber, pressurizing the chamber with about 3 to about 50 inches of mercury with an inert gas, and repeating until the product is degassed of the sterilent gas. Alternatively the step of degassing the product may be accomplished by evacuating the chamber, preferably down to a pressure in the range of 3 to 7 inches of mercury and pulsing the chamber with heated inert gas, preferably about 5 to about 9 inches of mercury worth of gas pressure. This step may be further accomplished by injecting the chamber with warm air. Warm air refers to air that is typically higher than room temperature. The exact temperature is not critical and depends on the specific article being sterilized and the sterilent gas.

Other steps may include evacuating the chamber, preferably to a pressure of about 1 to about 3 inches of mercury after the dwell time and pulsing in steam and/or heated Nitrogen into the chamber prior to degassing the product of sterilent gas. It can be advantageous to perform real-time monitoring of the concentration of ethylene oxide gas in the headspace in conjunction with the sterilization process.

Other aspects and advantages will become apparent from the following detailed description, taken in conjunction with the accompanying figures.

### DETAILED DESCRIPTION

A method for sterilizing products using ethylene oxide gas in conjunction with one or more of the following techniques is disclosed: steam pulses; steam conditioning; deep vacuum pulses with nitrogen; and, positive pressure pulses of inert gases. The entire sterilization process, which generally consists of conditioning, sterilizing, and degassing the product or article, is preferably performed in a single chamber. The sterilized product is releasable to the end user from an ethylene oxide residual standpoint at the completion of the process. The entire process takes preferably less than about 10 hours, but certain applications may require up to about 20 hours or more. The method of this invention is applicable to any product suitable for ethylene oxide sterilization. The method is especially applicable to medical device products.

The sterilization method of the present invention has several steps. Each step of the method has a specific purpose and yet works cooperatively with the other steps to thoroughly and speedily sterilize products. Preferably, each step is performed in the same chamber. However, in an alternate embodiment the entire sterilization process is performed in a continuous flow through process in which the material to be sterilized is moved through different steps in different chambers or equipment prior to completion at the end of the production line. For the preferred application of the method of the present invention, no special chamber is required. A conventional programmable industrial sterilization chamber that is equipped with a pump may be used. The pump is preferably capable of both introducing gasses into the chamber and pulling gas from the chamber to create a vacuum. The size of the chamber is not critical and depends on the scale of the load to be sterilized.

The first step in the process is referred to as the conditioning step. The purpose of the conditioning step is to raise the temperature of the product and/or introduce humidity into the chamber. Raising the temperature of the product and introducing humidity facilitates the sterilization reaction. This step may also be used to flush out air from the chamber. To begin the conditioning step, the product to be sterilized, referred to as the load, is placed in a sterilization chamber.

(Throughout the specification concentration ranges and pressure ranges are provided. These ranges are exemplary only and not intended to limit the scope of the invention. Those skilled in the art will recognize that different applications have different requirements.)

In the preferred embodiment the chamber is evacuated to a pressure of about 1 to 4 inches of mercury. After evacuation a combination of Nitrogen and steam are added into the chamber. Nitrogen is the preferred gas but any inert gas, such as helium, would be suitable. Any reference to Nitrogen includes any inert gas unless otherwise indicated. Preferably, the inert gas is heated above room temperature. Unless otherwise noted, heated inert gas is preferred throughout this specification when used.

In one aspect, first Nitrogen is injected (pushed) into the chamber then quickly removed (pulled) from the chamber, this action is commonly referred to as pulsing. Preferably, enough Nitrogen is pulsed to increase the pressure to about 2 inches of mercury and then the same amount of gas is pulled from chamber. The Nitrogen pulses may be done several times and it is preferable to do so. In one embodiment, the Nitrogen gas is heated. The preferred temperature range for the heated Nitrogen is 130 to 170° F. After the Nitrogen is pulled and the pressure is returned to near the value of the initial evacuation, i.e. a pressure of 1 to 4 inches of mercury.

In some embodiments, steam is pushed into the chamber after the inert gas is pulled out. Alternatively, the conditioning step can be performed without pulsing inert gas but only utilizing steam. The steam is pushed and pulled out of the chamber repetitively until the load is at the desired temperature and the humidity is at the desired level. In ethylene oxide sterilization, sterilization occurs at a faster rate at higher temperatures. In a typical application, the load may be sufficiently heated in less than 2 hours. In one embodiment, Nitrogen is injected over top of the steam. Preferably 10 to 20 inches of mercury of an inert gas, preferably Nitrogen, is added over top of the steam, which is believed to have the effect of forcing the steam towards the center of the load. In another embodiment a mixture of steam and inert gas are pulsed simultaneously. When the load is sufficiently conditioned, the steam and/or inert gas is again pulled out, preferably to a pressure near the initial evacuation, i.e. a pressure of about 1 to about 4 inches of mercury. At this point the sterilization step is initiated.

The sterilization step is initiated by injecting the sterilent gas into the chamber. The preferred sterilent gas is ethylene oxide. Unless otherwise indicated, any reference to ethylene gas is applicable to other sterilent gasses. Preferably, enough sterilent gas to raise the pressure about 9 inches of mercury is injected into the chamber. More or less can be injected depending on the type of sterilent gas and the product being sterilized. In a typical sterilization chamber, this would be about 400 to 550 mg/L of Ethylene oxide gas, but higher or lower concentration may be used. An optional method is to inject the gas at a reduced rate than conventional processes. Rates in the range of 0.1 to 0.2 inches per minute allow the gas to more fully vaporize and gain more sensible heat, which allows for a reduced dwell time.

The load is held in the chamber until the product is sterilized. The amount of time the load is held, often referred to as the dwell time, varies depending on the product being sterilized. An inert gas overlay or inert gas blanket (also referred to as overpressure) is added to the headspace of the sterilization chamber during the dwell. Preferably Nitrogen gas overpressure is added immediately following the injection of ethylene oxide and the pressure is maintained for the duration of the dwell period. The amount of inert gas overpressure is preferably in the range of about 5 to about 15 inches of mercury, most preferably around 10-12 inches of mercury. Generally, the more overpressure added to the headspace of the chamber, the lower the concentration of sterilent gas required and the less dwell time required to complete the sterilization.

It is believed that the inert gas overlay dynamically generates a greater surface-to-center pressure gradient on the load and shifts the highest concentration from the surface of the load towards the inside of the load. This has the effect of assisting the sterilent gas penetration into the center of the load and enhancing the uniformity of concentration distribution, thereby ensuring complete sterilization. It is also believed that the overpressure or overlay drives the steam or heated water vapor into the center of load thereby driving both heat and Ethylene oxide into the most difficult or densest areas of the product packaging configuration. This dynamic speeds what is normally considered a conventional conduction heat transfer. The presence of moisture is critical to the Ethylene oxide lethality mechanism for eradication of bacteria, yeasts & molds. The moisture coupled with the EtO are expedited to the niche areas where the bacterial flora reside thus allowing for quicker reaction time and therefore less dwell time needed to deliver the sterility necessary for the end product.

One advantage of the present invention is that the dwell time for a typical sterilization is reduced by ⅓ to ⅔ of conventional processes.

Typically, real-time measurements of the concentration of sterilent gas in the headspace is monitored during the dwell time, although not required. The preferred method of measuring and monitoring the concentration in the headspace is disclosed in U.S. patent application Ser. No. 10/361508, which is hereby incorporated herein by reference. Measurements of the headspace concentration of ethylene oxide taken while performing the present invention show the concentration drop from 450 mg/L to 150 mg/L in a matter of minutes.

At the completion of the dwell period, the chamber is evacuated down to a pressure of 1 to 3 inches of mercury. An optional method is to evacuate the chamber at a reduced rate from conventional processes. Rates in the range of 0.1 to 0.5 inches per minute can enhance the residual kill. The preferred reduced evacuation rate is 0.33 inches per minute. Optionally, moisture, in the form of steam can also be injected in pulses into the chamber to aid in completing the sterilization reaction. Alternatively, heated Nitrogen may also be pulsed into the chamber, or a combination of both can be pulsed into the chamber.

When sterilization is complete the load is degassed. In general the degassing step is accomplished by evacuating the chamber and then re-pressurizing the chamber with inert gas. Preferably, the chamber is evacuated to a pressure of about 2 to 3 inches of mercury, and then re-pressurized with Nitrogen gas, preferably with enough gas to increase the pressure to about 3 to 55 inches of mercury. This step of evacuating and re-pressurizing the chamber can be repeated as many times as necessary to degas the product. Alternatively, the chamber can be evacuated to about 3 to 7 inches of mercury and pulsed with heated inert gas, preferably enough to raise the pressure about 5 to 9 inches of mercury. This step may also be repeated as necessary to degas the product. Lastly, the degassing step may include injecting the chamber with warm air. The product is released when the process has completed the validated cycle parameters. These parameters are identified and evaluated as a result of specific product and process experimental evidence to develop the exacting process parameters, which renders to the product the appropriate level of lethality and residual reduction.

Specific instructions for practicing the invention are provided in the following examples. These examples are merely illustrative and do not limit the invention in any way.

### EXAMPLE 1

The following procedure is used to sterilize pallets of product using EO as the sterilent gas.

Loading:

Place 2 product temperature probes in th epallet at the geometrical center. Drain Vacuum pump prior to cycle start. Drain Vacuum pump during Gas dwell. Verify that all biological indicators are present on the load prior to placement into the processing chamber.
   Additional:

Records Product temperature prior to loading chamber (Minimum of 74 F.). Product temperature will be recorded throughout cycle processing. Monitor gas concentration mg/l during Gas dwell. (Minimum of 150 mg/l). After achieving pressure set point approx. 10 minutes into Gas Dwell. Load temperature at the end of Humidity Dwell 103 F. or greater. Load temperature throughout Gas Dwell 105 F. or greater. Load temperature during After Vacuum and Gas Wash A 107 F. or greater. Load temperature during Gas Wash B & C 98 F. or greater. Humidity at the end of Humidity Dwell 60% or greater. Humidity during Gas Dwell 37% or greater.

| | | Set Point | Minimum | Maximum |
|---|---|---|---|---|
| Load Temperature | Temperature: | 74 F. | 74 F. | 140 F. |
| Process Temperature | | 130 F. | 120 F. | 140 F. |
| Initial Vacuum | Evacuate To: | 2.0″HgA | 2.5″HgA | 1.5″HgA |
| Approx. Rate: 1.0″ Min | Time: | 40 Min | 25 Min | 90 Min |
| Humidification | | 1.0″Hg-Rise | 0.5″Hg-Rise | 1.5″Hg-Rise |
| Approx. Rate: NA | Time: | NA | NA | NA |
| Steam Conditioning | Humidity To: | 3.0″HgA | 2.5″HgA | 3.5″HgA |
| | Evacuate To: | 2.5″HgA | 3.0″HgA | 2.0″HgA |
| | Dwell Time: | 60 Min | 50 Min | 80 Min |
| Humidity Dwell | Dwell Time: | 15 Min | 15 Min | 25 Min |
| | Maintain Pressure At: | 3.0″HgA | 2.5″HgA | 3.5″HgA |
| Gas Inject | Gas By Weight: | NA | NA | NA |
| Inject Type | Drum Change Allowed? Yes | | | |
| | Gas To: | 11.2″HgA | 10.7″HgA | 11.7″HgA |
| Approx. Rate: 0.5″/Min | Time: | 20 Min | 8 Min | 45 Min |
| Parametric Release | Gas Con.: | 150 MG/L | 150 MG/L | 542 MG/L |
| | Gas Dwell) Load Temperature: | F. | 105 F. | 140 F. |
| | (Gas Dwell) Load Relative Humidity: | % | 37% | % |
| Gas Dwell | Temperature: | 130 F. | 125 F. | 140 F. |
| | Time: | 1 Hrs 40 Min | 1 Hrs 39 Min | 1 Hrs 50 Min |
| Maintain Pressure With: | N2 At: | 23.2″HgA | 22.7″HgA | 25.9″HgA |
| After Vacuum | Evacuate To: | 2.5″HgA | 3″HgA | 2″HgA |
| Approx. Rate: .33″/Min | Time: | 65 Min | 58 Min | 180 Min |
| | Vacuum Hold Time: | NA | NA | NA |
| Gas Wash A | Inject To: | 3.0″HgA | 2.5″HgA | 3.5″HgA |
| Approx. Rate: 1″/Min | Time: | 1 Min | 0.3/9 Min | 2/30 Min |
| Inject Type: | Evacuate To: | 2.7″HgA | 3.2″HgA | 2.2″HgA |
| Steam | | | | |
| Approx. Rate: NA ″/Min | Time: | | | |
| | Vacuum Hold Time: | NA | NA | NA |
| Number of Repeats: | 4(5Total) | | | |
| Gas Wash B | Inject To: | 26.0″HgA | 25.5″HgA | 26.5″HgA |
| Approx. Rate: 1″/Min | Time: | 24 Min | 18 Min | 40 Min |
| Inject Type: | Evacuate To: | 3.0″HgA | 3.5″HgA | 2.5″HgA |
| N2 | | | | |
| Approx. Rate: 1″/Min | Time: | 24 Min | 18/139 Min | 40/240 Min |
| | Vacuum Hold Time: | NA | NA | NA |
| Number of Repeats: | 2(3Total) | | | |
| Gas Wash C | Inject To: | 26.0″HgA | 25.5″HgA | 26.5″HgA |
| Approx. Rate: 1″/Min | Time: | 24 Min | 18 Min | 40 Min |
| Inject Type: | Evacuate To: | 3.0″HgA | 3.5″HgA | 2.5″HgA |
| Air | | | | |
| Approx. Rate: 1″/Min | Time: | 24 Min | 18/323 Min | 40/560 Min |
| | Vacuum Hold Time: | NA | NA | NA |
| Number of Repeats: | 6 (7Total) | | | |
| Final Release | Release To: | 28.0″HgA | 27.5″HgA | NA ″HgA |
| Approx. Rate: 1.0″/Min | Time: | 26 Min | 20 Min | 60 Min |

### EXAMPLE 2

The following procedure is used to sterilize 30 pallets of product using EO as the sterilent gas.

Preprocessing:

Probes (Internal Temperature) will be placed in pallet #s 1, 8 and 15 prior to loading chamber. Temperature must be 75 F. If temperature is below 75 F., the load will be placed load in a preheating room to bring the temperature to specification. Plug in product thermocouples and place between cases in middle pallets 1 and 16. All loads will consist of 30 pallets.
   Loading:
All pallets will be loaded in descending order with pallets 1-15 on the right side of the chamber and 16-30 on the left side of the chamber.
   Other:
1) Parametric Release Criteria:
   1.1) A temperature probe will be placed in pallet #1 and pallet #16 (geometric centers) to monitor load temperature during ETO Gas dwell and steam temperature
1.2) EO Concentration must meet minimum requirement after N₂ injection.

2) Maximum temperature during washes is 150.

| | | Set Point | Minimum | Maximum |
|---|---|---|---|---|
| Load Temperature | Temperature: | 75 F. | 75 F. | N/A |
| Process Temperature | | 135 F. | 125 F. | 145 F. |
| Initial Vacuum | Evacuate To: | 2.0″HgA | 1.5″HgA | 2.5″HgA |
| Approx. Rate: 1.0″ Min | Time: | N/A | N/A | N/A |
| Nitrogen Wash | Humidity To: | N/A | N/A | N/A |
| | N₂ Inject To: | 12.0″HgA | 11.5″HgA | 12.5″HgA |
| Approx. Rate: N/A | Time: | N/A | N/A | N/A |
| Number of Repeats | | | | |
| One Total | Evacuate To: | 2.8″HgA | 2.3″HgA | 3.3″HgA |
| Approx. Rate: N/A | Time: | N/A | N/A | N/A |
| Humidification | | N/A″HgA | N/A″HgA | N/A″HgA |
| Approx. Rate: NA | Time: | NA | NA | NA |
| Steam Conditioning | Humidity To: | 2.8″HgA | 2.3″HgA | 3.3″HgA |
| | Evacuate To: | 2.2″HgA | 1.7″HgA | 2.7″HgA |
| | Dwell Time: | 90 Min | 85 Min | 120 Min |
| Humidity Dwell | Dwell Time: | 10 Min | 10 Min | 15 Min |
| | Maintain Pressure At: | 2.8″HgA | 2.3″HgA | 3.3″HgA |
| Gas Inject | Gas By Weight: | NA | NA | NA |
| Inject Type | Drum Change Allowed? Yes | | | |
| | Gas To: | 12.9″HgA | 12.4″HgA | 13.4″HgA |
| Approx. Rate: 1.0″/Min | Time: | N/A | N/A | N/A |
| Parametric Release | Gas Con.: | 550 MG/L | 350 MG? | 750 MG/L |
| Gas Dwell | Temperature: | 135 F. | 130 F. | 145 F. |
| | Time: | 2 Hrs 30 Min | 2 Hrs 30 Min | 2 Hrs 35 Min |
| Maintain Pressure With: | Inert At: | 25.0″HgA | 24.0″HgA | 28.0″HgA |
| After Vacuum | Evacuate To: | 3.0″HgA | 2.0″HgA | 4.0″HgA |
| Approx. Rate: 0.3″/Min | Time: | N/A | N/A | N/A |
| | Vacuum Hold Time: | N/A | NA | NA |
| Gas Wash A | Inject To: | 50″HgA | 49.5″HgA | 50.5″HgA |
| Approx. Rate: 2.0″/Min | Time: | N/A | N/A | N/A |
| Inject Type: | Evacuate To: | 3.0″HgA | 2.5″HgA | 3.5″HgA |
| Inert | | | | |
| Approx. Rate: 0.4″″ Min | Time: | N/A | N/A | N/A |
| | Vacuum Hold Time: | 15 Min | 15 Min | 20 Min |
| Number of Repeats: | Four total | | | |
| Gas Wash B | Inject To: | 12.0″HgA | 11.5″HgA | 12.5″HgA |
| Approx. Rate: 2.0″/Min | Time: | N/A | N/A | N/A |
| Inject Type: | Evacuate To: | 1.0″HgA | 0.5″HgA | 1.5″HgA |
| Air | | | | |
| Approx. Rate: 0.4″/Min | Time: | N/A | N/A | N/A |
| | Vacuum Hold Time: | 5 Min | 5 Min | 10 Min |
| Number of Repeats: | One Total) | | | |
| Gas Wash C | Inject To: | 25.0″HgA | 24.5″HgA | 25.5″HgA |
| Approx. Rate: 2.0″/Min | Time: | N/A | N/A | N/A |
| Inject Type: | Evacuate To: | 1.5″HgA | 1″HgA | 2″HgA |
| Air | | | | |
| Approx. Rate: 0.4″/Min | Time: | N/A | N/A | N/A |
| | Vacuum Hold Time: | 5 Min | 5 Min | 10 Min |
| Number of Repeats: | One Total | | | |

1. A method for sterilizing industrial products comprising the steps of:
conditioning an industrial product to be sterilized by placing the product in a chamber, evacuating the chamber, pulsing steam and/or heated inert gas into the chamber, and re-evacuating the chamber;
injecting a sterilent gas into the chamber;
introducing an overpressure of inert gas into the chamber;
holding the product in the chamber until the product is sterilized;
degassing the product.

2. The method for sterilizing industrial products of claim 1 wherein the heated inert gas is Nitrogen and wherein the sterilent gas is ethylene oxide.

3. The method for sterilizing industrial products of claim 1 further comprising the step of evacuating the chamber after holding the product in the chamber and pulsing in steam and/or heated inert gas into the chamber.

4. The method for sterilizing industrial products of claim 3 wherein the heated inert gas is Nitrogen and wherein the sterilent gas is ethylene oxide.

5. The method for sterilizing industrial products of claim 4 wherein the evacuating the chamber results in the pressure in the range of 1 to 3 inches of mercury.

6. The method for sterilizing industrial products of claim 3 wherein the step of degassing the product is accomplished by evacuating the chamber, pressurizing the chamber with 3 to 50 inches of mercury with an inert gas, and repeating until the product is degassed.

7. The method for sterilizing industrial products of claim 3 wherein the step of degassing the product is accomplished by evacuating the chamber down to 3 to 7 inches of mercury and pulsing the chamber with 5 to 9 inches of heated inert gas.

8. The method for sterilizing industrial products of claim 6 and 7 wherein the wherein the step of degassing the product is further accomplished by injecting the chamber with warm air.

9. The method for sterilizing industrial products of claim 5 further comprising the step of real-time monitoring the concentration of ethylene oxide gas in the headspace.

10. The method for sterilizing industrial products of claim 9 wherein the step of degassing the product is accomplished by evacuating the chamber, pressurizing the chamber with 3 to 50 inches of mercury with Nitrogen, and repeating until the product is degassed.

11. The method for sterilizing industrial products of claim 9 wherein the step of degassing the product is accomplished by evacuating the chamber down to 3 to 7 inches of mercury and pulsing the chamber with 5 to 9 inches of heated Nitrogen.

12. The method for sterilizing industrial products of claim 10 and 11 wherein the wherein the step of degassing the product is further accomplished by injecting the chamber with warm air.

13. The method of claim 6 wherein evacuating the chamber as a part of degassing the product is performed at a rate in the range of 0.1 to 0.5 inches per minute.

14. A method for sterilizing industrial products comprising the steps of:
conditioning an industrial product to be sterilized by placing the product in a chamber, evacuating the chamber, pulsing steam and/or heated inert gas into the chamber, and re-evacuating the chamber;
injecting ethylene oxide gas into the chamber;
introducing 5 to 15 inches of mercury of Nitrogen overpressure into the chamber;
holding the product in the chamber while the product is sterilized;
evacuating the chamber to a pressure of 1 to 3 inches of mercury;
pulsing in steam and/or heated Nitrogen into the chamber; and
injecting the chamber with warm air.

15. The method of claim 14 wherein evacuating the chamber to a pressure of 1 to 3 inches of mercury is done at a rate of 0.1 to 0.5 inches per minute.

16. The method for sterilizing industrial products of claim 15 wherein the step of pulsing in steam and/or heated Nitrogen into the chamber is repeated one or more times.
